(19) United States
(12) Patent Application Publication
(10) Pub. No.: US 2003/0068252 A1
(43) Pub. Date: Apr. 10, 2003

(54) Crystal nucleating chip

(76) Inventor: Tu Lee, Monmouth Junction, NJ (US)

Correspondence Address: Richard C. Litman, LITMAN LAW OFFICES, LTD., P.O. Box 15035, Arlington, VA, 22215, US

(21) Appl. No.: 09/971,653

(22) Filed: Oct. 9, 2001

(51) Int. Cl. G01N 33/53; B32B 27/12; G01N 33/543

(52) U.S. Cl. 422/99; 435/7.1; 436/518

(57) ABSTRACT

The Crystal Nucleating Chip is suitable for use as a high-throughput, low cost, disposable mono-layer array type device, specifically for simultaneously forming crystals of inorganic or organic solute under the inducement of an array of unique SAM's chemical functional head groups and determining their effects on crystal polymorphism and crystal habit of the solute in solutions. The array is a substrate divided into a pattern of pads that directly or indirectly support a number of test compounds each with a specific structure and functionality. A method of using the invention involves bringing the array into contact with a solution in order to promote potential crystallization of the solute. The method further allows for qualitatively determining polymorphism, i.e., crystal formation, and crystal habit of the specific test compounds, e.g. SAMs, through a variety of spectroscopic and microscopic techniques.

### BACKGROUND OF THE INVENTION

1. Field of the Invention

The present invention relates to a high through-put self-assembled mono-layered (“SAM”) crystal nucleating device, specifically for simultaneously forming crystals of inorganic or organic solute induced by an array of unique SAM's chemical functional groups and determining their specific effects on crystal polymorphism and crystal habit in solutions.

2. Description of the Related Art

Traditional techniques for growing crystals primarily occur in test tubes where large volumes of solute (material which will be crystallized), solvent, anti-solvent and/or foreign aids, e.g. surfactants and polymers, are mixed causing crystal precipitation. The general techniques are particularly inefficient because trial-and-errors at a bench scale are needed to test solvents, anti-solvents and/or foreign materials with a particular functional group for the promotion and the control of crystallization of the solute in a liquid solution. Even the automation of these trial-and-error techniques is still time consuming, involving large amounts of expensive compounds of which many are toxic and sometimes unstable. As a result, the traditional techniques are inappropriate and non-economical when screening solvents suitable for crystallization, identifying compatible polymers for co-crystallization, controlling crystal habit and polymorphs, i.e., different forms of the same compound.

Scientists are developing techniques that remove some of the limitations discussed above. The techniques primarily involve manipulation of molecules at the micro-level aimed at ways to develop uniform, thin layers of compounds upon which reactions and crystallization may occur. These thin layers known as “self-assembled monolayer” (37 SAMs”), to those in the art, refer to a relatively ordered assembly of molecules spontaneously chemisorbed on a surface, in which the molecules are oriented approximately parallel to each other and roughly perpendicular to the surface.

Each of the molecules generally includes a functional tail group that adheres to the surface, a separate functional head group that is pointing upward which controls the crystallization event, and a portion that interacts with neighboring molecules in the monolayer to form the relatively ordered array.

Aizenberg, J.; Black, A. J.; and Whitesides, G. M., “Oriented Growth of Calcite Controlled by Self-Assembled Monolayers of Functionalized Alkanethiols Supported on Gold and Silver,” J.Am. Chem. Soc., 121, 4500-4509 (1999) describes a method of orienting the nucleation of calcite controlled by self-assembled monolayers of —terminated alkanethiols (HS(CH₂)ₙX) supported on metal films and the ability to control the nucleation of crystal growth. This method only applies to crystallization of calcium carbonate in an aqueous solutions under the direction of alkanethiols terminated with functional head groups, such as COO—, SO3—, PO32—, OH, N(CH₃)₃⁺, and CH3. It describes the test of one type of SAM at a time and not the test of a plurality of SAMs simultaneously to form calcium carbonate crystals with different polymorphs and habits in the same solution. The Aizenberg et al. process, therefore, does not describe a crystal nucleating chip composed of a matrix of discrete pads where each pad supports a unique SAM simultaneously used for crystallization, crystal polymorphism and crystal habit. Finally, Aizenberg et al. does not describe a method of using or making the crystal nucleating chip.

A similar article by Aizenberg, J.; Black, A. J.; and Whitesides, G. M., “Control of Crystal Nucleation by Patterned Self-Assembled Monolayers,” Nature, 398, 495-498 (1999) describes a route to crystal formation using micropatterned self-assembled monolayers to control simultaneously the density and pattern of nucleation events, and the size and orientation of the growing crystals. Like the process in the previous article, this method only applies to crystallization of calcium carbonate in an inorganic calcium carbonate solution on a monolayer of alkanethiols where one type of SAM is tested at a time, not a plurality of SAMS simultaneously used to form crystals in solution. Aizenberg et al., therefore, does not describe a crystal nucleating chip composed of a matrix of discrete pads where each pad contains a unique SAM simultaneously used for crystallization, crystal polymorphism and crystal habit. Finally, Aizenberg et al. does not describe a method of using or making the crystal nucleating chip.

A similar article by Kang J. F.; Zaccaro, J.; Ulman, A.; and Myerson, A., “Nucleation and Growth of Glycine Crystals on Self-Assembled Monolayers on Gold,” Langmuir, 16, 3791-3796 (2000) describes a method of only modifying the crystal habit of glycine controlled by self-assembled monolayers of thiol compounds: 4′hydroxyl-4-mercaptobiphenyl,4-(4-mercatophenyl)-pyridine, and their mixed SAMs with 4′-methyl-4-meercaptobiphenyl supported on gold surface in glycine containing aqueous solution. Kang et al. does not describe any crystal nucleating chips composed of a matrix of discrete pads where each pad contains a unique SAM or mixed SAMs simultaneously used for crystallization, crystal polymorphism and crystal habit. Again, Kang et al. does not describe a method of using or making the array.

The previous references as described focus on making effective self-assembled monolayers but do not describe an array device containing a matrix of discrete pads simultaneously testing a plurality of mixed SAMs for crystal polymorphism and habit. Now, another method is reported using light to direct the simultaneous synthesis of many different biological compounds on an array. Fodor, S. P. A.; Leighton Read, J.; Pirrung, M. C.; Stryer, L.; Lu, A. T.; and Solas, D., “Light-Directed Spatially Addressable Parallel Chemical Synthesis,” Science, 251, 767-773 (1991), describes a method for synthesizing an array of 1024 peptides and its interaction with a monoclonal antibody. The article further describes that oligonucleotide arrays may be produced by light-directed synthesis for use in detecting complementary sequences in DNA and RNA and of other biological receptors. Although Fodor et al. describes a method to synthesize an array of peptides, which may be extended to surface treatment, the Fodor et al. method does not describe a crystal nucleating chip composed of a matrix of discrete pads where each pad contains a unique SAM simultaneously tested used in an inorganic or organic solution for crystallization, crystal polymorphism and habit.

Aizenberg, J., “Patterned Crystallization On Self-Assembled Monolayers with Integrated Regions of Disorder,” J. Chem. Soc., Dalton Trans., 3963-3968 (2000), describes new methods of controlling the pattern of SAMs using mixed metal substrates. By using a mix of metal substrates, localized regions of disorder in SAMs are generated at the edges between the different metals, and thus alter the pattern of the growth of SAMs, in an inorganic solution. Aizenberg's method focuses on the patterning of growing identical SAMs on the substrate in an inorganic solution, not on attempting to simultaneously use a plurality of mixed SAMs for forming crystals in diverse solutions, including organic solutions. Finally, Aizenberg does not describe a crystal nucleating chip composed of a matrix of pads where each pad contains a unique SAM simultaneously used for crystallizaton, crystal polymorphism and habit. Therefore, the claimed invention is distinguishable from the above method.

A variation of the previous reference is found in U.S. Pat. No. 6,114,099, issued Sep. 5, 2000, to Yanjing Liu and Guy A. Schick. This patent discloses a method for making a multi-layered, multi-patterned molecular self-assembly. Liu applies a mask material to pattern a layer of photo-resist and exposes the photo-resist to various wavelengths of light, including UV light, where the exposed substrates are capable of selectively adsorbing at least a first compound thus forming the first layer of SAMs. Liu uses mask material and inert blocking compounds to selectively add in the vertical plane (Z-axis), a second compound to the first layer thus forming a first bi-layer with the process repeated as desired until a multi-layered self-assembly is formed. This multi-layer structure is necessary to achieve the desired optical and electric functional effects.

Liu does not describe the Lee invention, which is a crystal nucleating chip structurally composed of a monolayer of chemically unique test compounds with common chemical anchoring functional tail groups but different terminal functional head groups where each test compound is disposed on a specific pad. Further, Liu does not teach the use of a horizontally oriented crystal nucleating chip in solution where the test compounds are simultaneously used for crystallization, crystal polymorphism and habit. Therefore, the claimed invention is distinguishable from the above Liu method.

Finally, in U.S. Pat, No. 5,961,934, issued to Leonard Arnowitz and Emmanuel Steinberg, and U.S. Pat. No. 5,641,681, issued to Daniel C. Carter, both disclose a variety of traditional mechanical devices containing enclosed and atmospherically sealed crystallization chambers to grow crystals under gravitational and gravity free environments. Neither the Carter nor the Arnowitz et al. patents disclose a crystal nucleating chip composed of a matrix of pads where each pad contains a unique self-assembled monolayer (“SAM”) simultaneously used for crystallization, crystal polymorphism and habit. Further, the crystal nucleating chip does not use any type of enclosed or atmospherically sealed crystallization chamber.

None of the above inventions and patents, taken either singularly or in combination, is seen to describe the instant invention as claimed. Thus a crystal nucleating chip solving the aforementioned problems is desired.

### SUMMARY OF THE INVENTION

The invention is a crystal nucleating chip, which is used as a high-throughput, low cost, disposable mono-layer array type device, specifically for simultaneously forming crystals of inorganic or organic solute under the inducement of an array of unique SAM's chemical functional (head) groups and determining their specific effects on crystal polymorphism and crystal habit in solutions. The array is a substrate divided into a pattern of pads that directly or indirectly support a number of test SAMs each with a specific structure and functionality. A method of the invention involves bringing the array into contact with a saturated solution in order to promote potential crystallization and allowing for determination of the potential polymorphism (different types of crystal lattice structure of the same compound) and crystal habit of the solutes. The method may be automated and the use of the array facilitates the process of qualitatively determining polymorphism and crystal habit udner the direction of the terminal functional head groups of SAMs through a variety of spectroscopic and microscopic techniques.

Accordingly, it is a principal object of the invention to provide an easy to use and disposable crystal nucleating chip for simultaneously forming crystals on a plurality of test compounds, i.e., the terminal functional head groups on SAMs, in solution and determining their effects on the related crystal polymorphism and crystal habit properties, i.e., form, size, melting point, flowability, compactibility, and friability, so that solvents, antisolvents or foreign aids (such as surfactants and polymers) with the same or similar functional head group can be chosen.

It is another object of the invention to provide high-throughput identification of the formed crystals, crystal polymorphism and crystal habit through integration with X-ray diffraction, infrared spectroscopy/imaging, Raman spectroscopy/imaging, electron microscopy, atomic force microscopy, secondary ion mass spectroscopy and optical microscopy techniques.

It is a further object of the invention to be used in an automated process.

It is another object of the invention to provide unique seed crystals which can be harvested from the crystal nucleating chip and then added to a separate liquid bulk solution to promote growth of a specific crystal polymorph or habit in a large-scale production.

Still another object of the invention is to provide an efficient and cost-effective method for using the crystal nucleating chip at various experimental conditions, such as, temperature, atmospheric pressure, concentration of solute and solvent, to promote crystallization and determine the effect of specific chemical functional groups of SAMs on crystal polymorphism and habit of the solute in solution.

These and other objects of the present invention will become readily apparent upon further review of the following specification and drawings.

### BRIEF DESCRIPTION OF THE DRAWINGS

FIG. 1 is an environmental, perspective view of a crystal nucleating chip according to the present invention.

FIG. 2 is a perspective view of an individual pad.

FIG. 3A is a side illustration of an individual pad with an intermediate support layer.

FIG. 3B is a side illustration of an individual pad.

FIG. 4 sets forth the basic chemical structure of the test compound.

FIG. 5 is a side view of the substrate surface according to a first embodiment of the invention.

FIG. 6A is a side view of the substrate surface according to a second embodiment of the invention.

FIG. 6B is an environmental, perspective of a crystal nucleating chip with a plurality of different test compounds (SAMs) according to the present invention.

FIG. 7 is a side view of the device in use.

FIG. 8 is a block diagram setting forth a method for using the device.

FIG. 9A is a block diagram of a first method for making the device.

FIG. 9B is a block diagram of a second method for making the device.

FIG. 9C is a block diagram of a third method for making the device.

Similar reference characters denote corresponding features consistently throughout the attached drawings.

### DETAILED DESCRIPTION OF THE PREFERRED EMBODIMENTS

The present invention relates to a high-throughput, self-assembled monolayered (SAM) crystal nucleating device, specifically for simultaneously forming crystals of inorganic or organic solute under the inducement of an array of unique SAM's chemical functional head groups and determining their specific effects on crystal polymorphism and crystal habit of organic and inorganic solutes in solution through molecular registry.

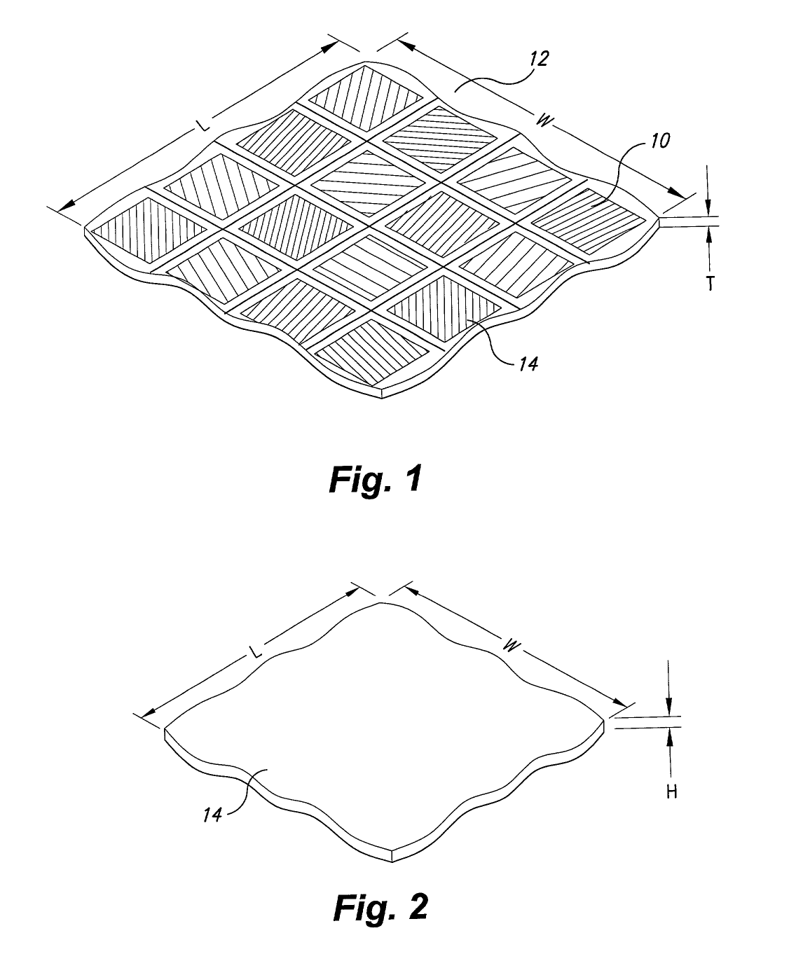

The present invention, referring to FIG. 1, is a Crystal Nucleating chip in the form of an array type device (“Array” or “Device”) generally designated at 10 of width (w), length (l) and thickness (t). The dimensions and shape of the device can vary depending on the needs of the activities to be performed. More specifically, the Device is designed to form, simultaneously, crystals of the solute under the inducement of an array of SAM's terminal functional head groups in the same solution. The Device is used as a platform for bonding and supporting a diverse group of test compounds, such as SAMs, where each compound is composed of a unique chemical chain terminating with different organic and inorganic functional groups but having a chemically common anchoring group.

The Array is formed on a horizontally oriented substrate or surface which may be metallic or non-metallic in nature. More particularly, the substrate generally is comprised of the following material: a single crystal silicon, silica, plastic composite, ceramic or a metal. The substrate is divided to form a matrix or pattern 12 of a plurality of equally spaced pads 14, where a minimum of two pads define the area of the array. The array can be any desired size, thickness or geometric shape as indicated above. Similarly, the shape and dimensions of a pad can vary as well. Generally, the length and width of a pad can range from 1 micro m up to an unlimited size (micro m). If an array is made by the inking and stamping method, the dimensions of the pads are limited by the size of the stamp's patterns. Further, the pad is preferably square shaped with a length and a width each preferably ranging from one (1) to 100 micro m according to FIG. 2. Since a pad is a defined area of the overall substrate, the thickness of the pad can vary with a minimum thickness being the same as the substrate or greater if an additional intermediate support layer is deposited on the substrate. In addition, the separation between each pad is known as the “feature.” The feature can range from as small as 0.2 micro m (200 nm) up to an unlimited size. Since pads are used in lieu of cells, no vertical walls exist between the pads. This information about pattern and feature dimensions is generally discussed in Kumar, A.; Biebuyck, H. A.; Whitesides, G. M., “Patterning Self-Assembled Monolayers: Applications in Materials Science,” Langmuir, 10, 1498-1511 (1994), the disclosure of which is hereby incorporated by reference in its entirety.

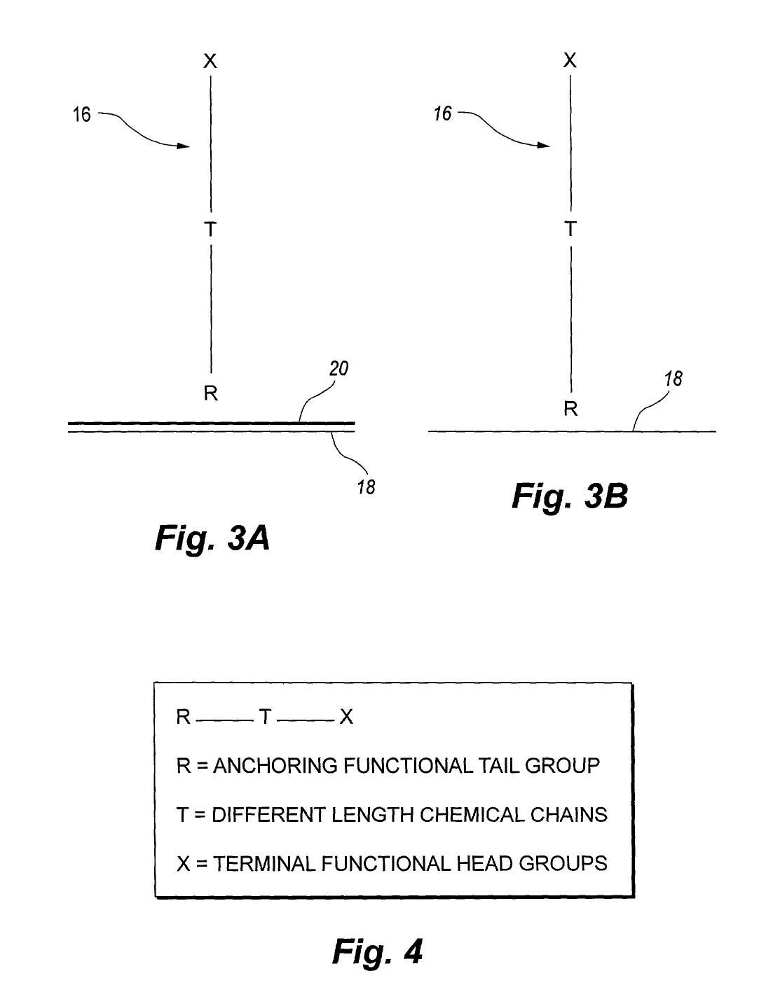

A test compound, i.e., a chemical chain, 16 is either indirectly bonded to the area of the substrate 18 by using an intermediate support layer 20 as in FIG. 3A or directly bonded to the substrate 18 as in FIG. 3B which defines the pad. The bond formed is generally covalent or ionic in nature depending on the chemistry of a test compound and the substrate. The test compound is shown in FIG. 4. Each test compound 16 is a chemical chain of the general form R-T-X where R is an anchoring tail group having a unique functionality. The R group includes thiols, aminos, proteins or other functional groups with the capability of directly or indirectly binding to the substrate. T represents an intermediate chemical group of different length chemical chains including, but not limited to, a CₙHₘ chain, CₙHₘOₚ chain, CₙHₘN_{q} chain, Si(n)Hₘ chain, Si(n)HₘOₚ chain, Si(n)HₘN_{q} chain, a polypeptide chain (contains C, H, O and N), polynucleotide chain (contains C,H, O, N, and P), SiO—SiO—SiO chain, and organometallic chains. X represents a variety of terminal organic and inorganic functional groups of interest including, but not limited to, —CH₃, —C₆H₅, —OH, —COOH, —COCH₃, —COC₂H₅, —CO(CH₃)₃, —COCH(CH₃)₂, —C(CH₃)₂OH, —CH(CH₂)₃O, —COOCH₃, —COOC₂H₅, —CH(CH₃)₂, —H, —CH₂(C₆H₅), —NH₃+, —CHCH₃OH, —SH, —CONH₂, —SCH₃, —NHC(NH₂)₂+, —C₃N₂H₄+, and —C₈NH₆.

As shown in FIG. 4, R and X are separately bonded to opposite terminal ends of the specific chemical chain, T, resulting in the structure denoted by R-T-X. R, X, the length of the chemical chain, T, and the chemical composition of T can, and will likely, vary for each pad in order to provide maximum flexibility for forming crystals and determining crystal polymorphism and habit of the solute.

The particular anchoring group, R, generally is directly attached to the substrate through a covalent or ionic bond. Indirect attachment may occur, however, when weak electron affinity exists between the overall test compound, i.e., chemical chain, and the substrate. This situation generally requires the use of an intermediate support layer of a metal, preferably including gold, silver, titanium, palladium, or other metals capable of bonding with the chemical chain and, in particular, R. More specifically, gold and silver intermediates are preferred when R is a thiol group. Titanium and other metallic surfaces are preferred when R is an amino group. When used, the intermediate support layer 20 is deposited on the substrate 18, and thus situated between the substrate and the anchoring group, R, of the test compound. In this orientation, the intermediate support layer 20 functions to bind the exposed areas of the substrate, i.e., the pads, while simultaneously binding with the particular anchoring group R of the test compound. As a result, the indirect bond between the test compound and substrate (via the intermediate support layer) is stronger than the attractive forces present in the intended environment of use on the surface of the chemical chain and attached terminating functional group, X, in solution. These types of intermediate support layers are described above in the Kumar et al. article.

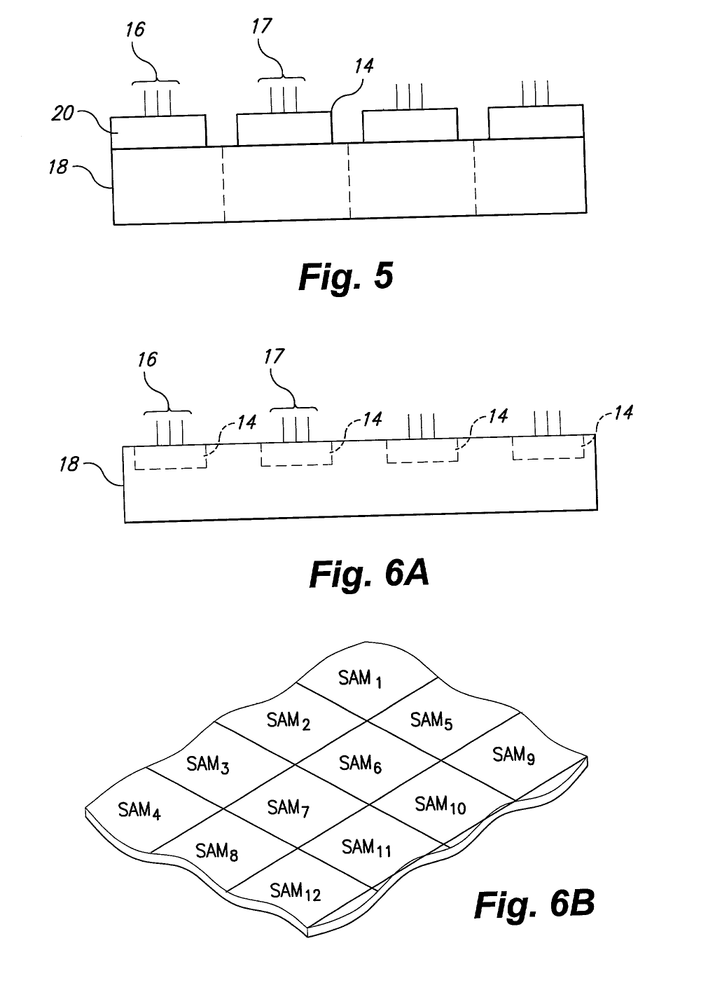

For clarification, the Crystal Nucleating Chip has a matrix of pads. A test compound composed of a chemical chain with a unique chemical structure, including a common anchoring group R, is bonded to the pad to form a self assembled monolayer (“SAM”). As a result, the chemistry of each SAM associated with a particular pad is different from the chemistry of the other SAMs and related pads except for the use of a common anchoring group. At least one chemical chain is bonded to each pad and, generally, a plurality of identical chemical chains are bonded to each pad. The chemical chains on each pad are generally vertically oriented, and thus parallel to each other and generally perpendicular to the pad. This orientation promotes the formation of a strong covalent or ionic bond. The group of test compounds are bonded onto each pad either directly, according to FIG. 5, or indirectly,according to FIG. 6A. Therefore, an Array according to FIG. 6B is formed with chemically distinct self assembled monolayers bonded to each pad as defined above.

The array, referring to FIG. 7, is turned upside down, resting on two vertical supports 22 with the array 10 being horizontally oriented and, preferably, the terminal functional groups, X, pointing vertically downward while being completely immersed in a receptacle 24 containing a solution 26. This preferred orientation of the terminal functional groups is sought to promote crystal formation solely on the terminal functional groups while avoiding crystals formed in free solution via homogeneous nucleation to deposit on the complete SAM by gravity. This immersion technique is described in Aizenberg, J., Black, A. J., Whitesides, G. M., “Control of Crystal Nucleation By Patterned Self-Assembled Monolayers,” Nature, 398, 495-498 (1999), which is hereby incorporated by reference in its entirety.

The immersion time in solution is based on the reactants and, specifically, will vary depending on the chemistry of the specific solution and the diverse chemical composition of the SAMs on the pads. Further the temperature of the immersed array and receptacle can be modified by application of conventional heating or cooling techniques. However, in a preferred embodiment, the solution is 25 to 100% saturated and the Array is operated in either an aqueous or organic solution from 25 to 60 degrees celsius. The crystallization reaction can be carried out under various atmospheric pressure conditions as needed and in atmospheric conditions of different chemistries. In the preferred embodiment, the reaction occurs in ambient air at a standard pressure of 1 atm.

After the specific reaction time is completed, the array is removed from the solution. The Array is rinsed and dried.

The next step is to analyze each pad for the formation of crystals. More specifically, the analysis focuses on whether the solution has crystallized out on a specific SAM. Any crystals, which have formed, are examined to determine crystal polymorphism and crystal habit. This analysis is performed using a variety of optical and electronic spectroscopy techniques, including 1) optical, electronic, Raman and atomic force microscopy; 2) Raman, Fourier transform infrared, X-ray photo-electron spectroscopy; 3) X-ray diffraction; and 4) differential scanning calorimetry. If crystallization occurred and the need exists, the crystals may be used for further study as well as seed crystals for large-scale production.

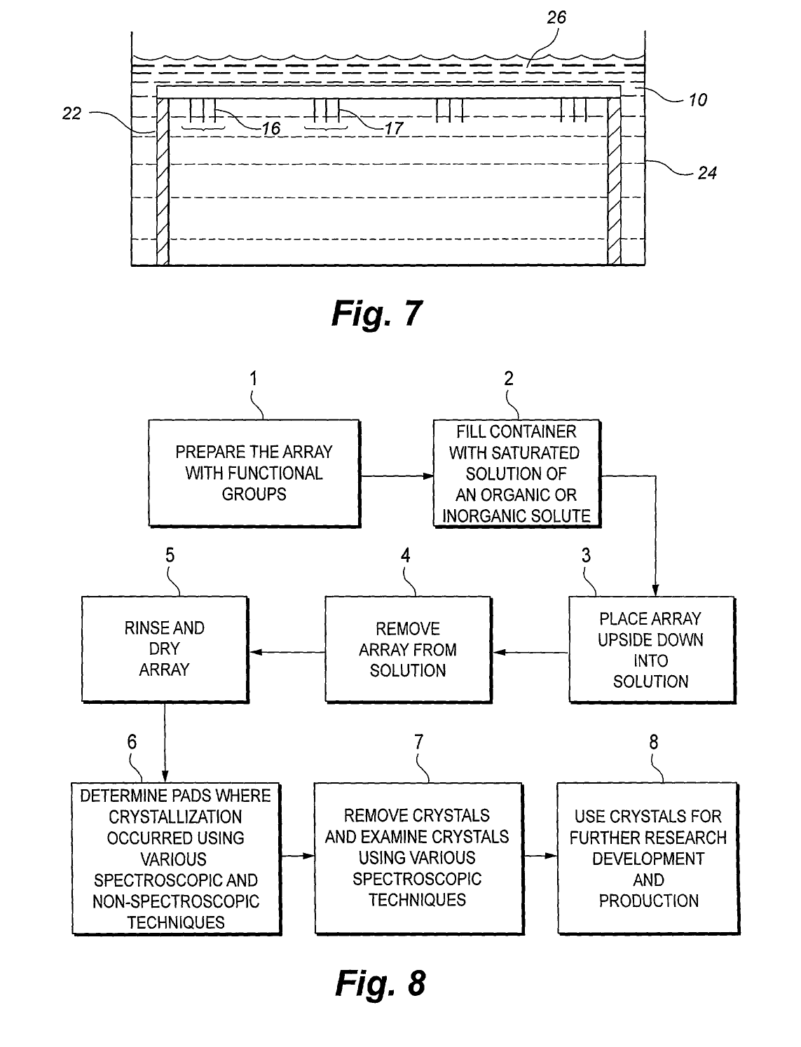

FIG. 8 depicts the above process for using the Array. The Device may be used manually or automatically with a variety of robotic instruments to achieve the same result. For example, the Array can be immersed, removed and rinsed by an automatic arm. Multiple receptacles with different temperatures, pressures and concentrations of solute can be automatically controlled and each pad of the array can be scanned and characterized automatically by spectroscopic and microscopic techniques.

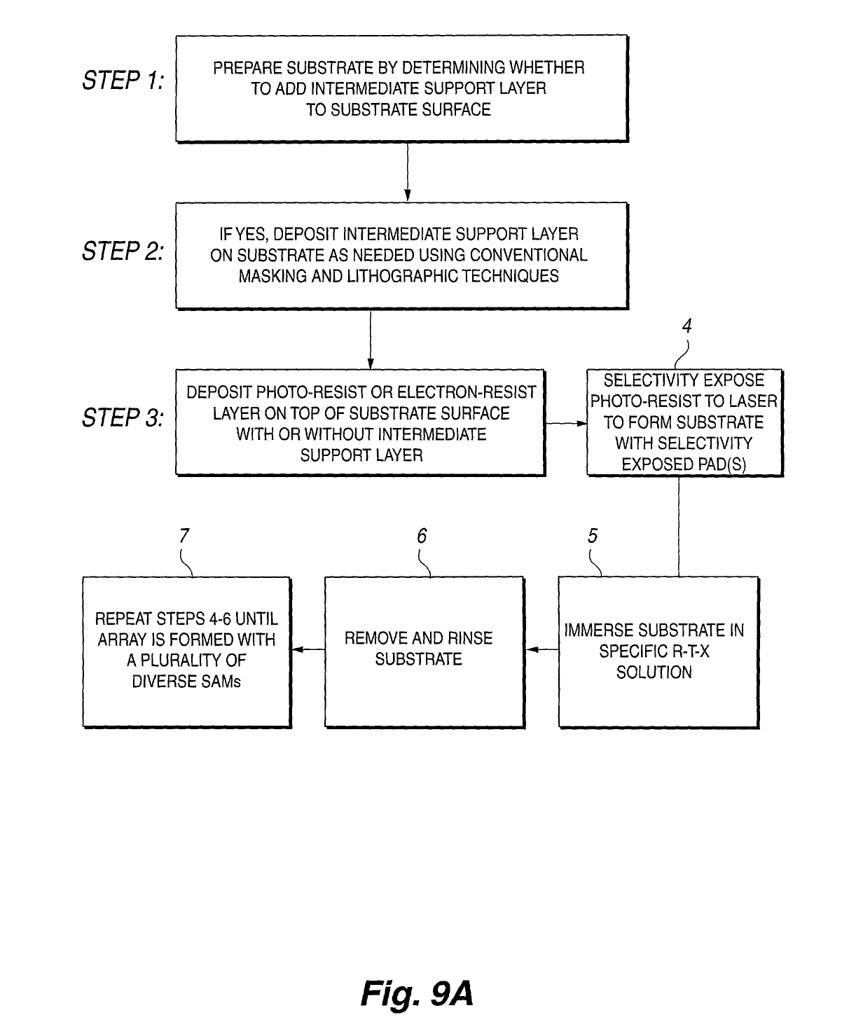

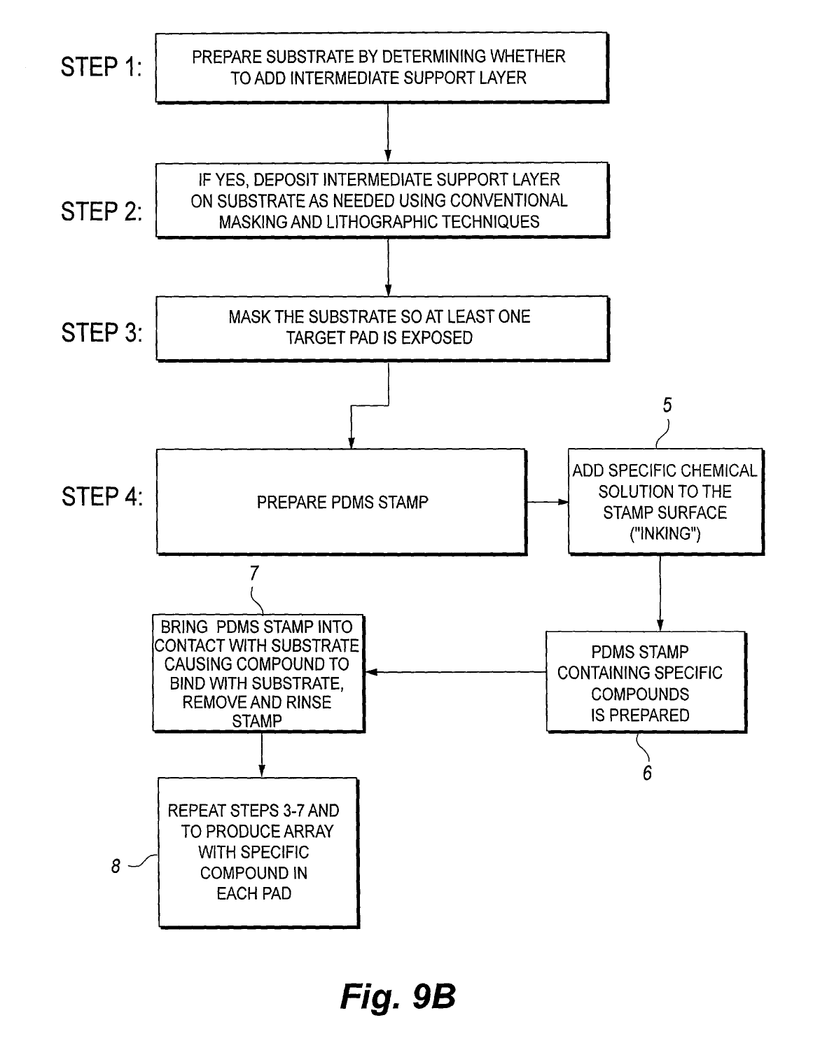

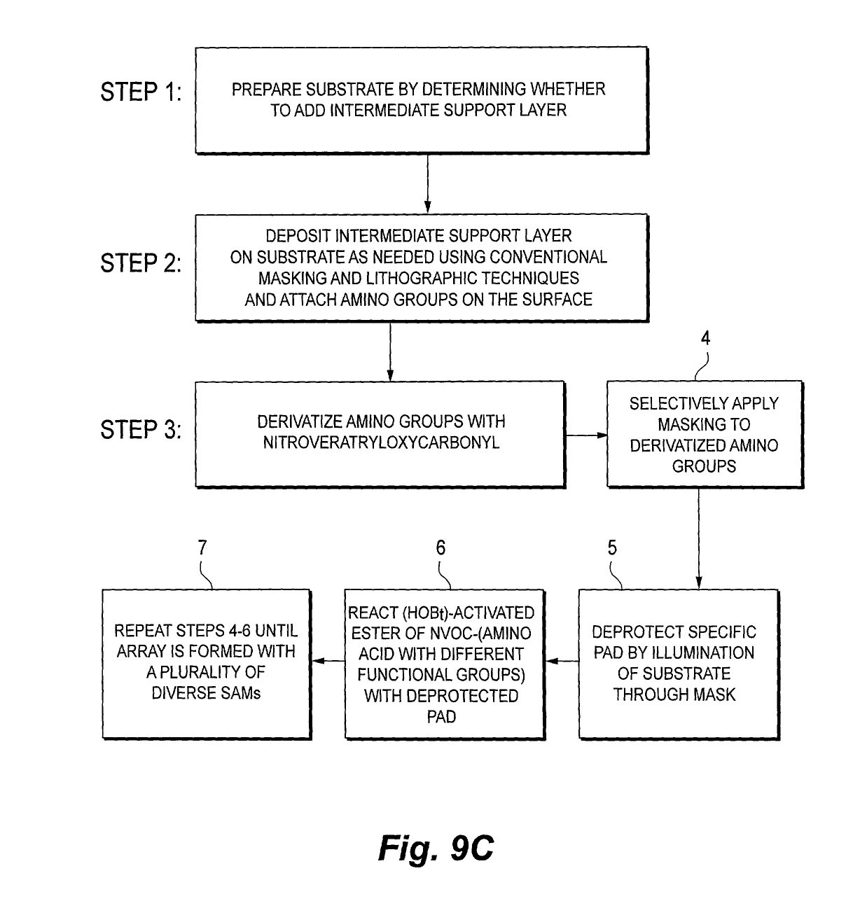

The array is made by one of three processes referred to in FIGS. 9A-9C. The first step, which is common to all three processes, involves taking the metallic or non-metallic substrate and, as needed, depositing on the substrate an intermediate support layer of gold, silver, titanium, palladium or other metallic materials. The intermediate support layer provides a surface permitting the indirect binding of the chemical chains to the substrate at particular locations on the substrate, i.e., the pads. As discussed, the determination to use the intermediate support layer is made by evaluating the affinity of the complete chemical chain and anchoring group, R, to form an ionic or covalent bond with the substrate absent an intermediate support layer. If a direct bond between the anchoring group, R, of the chemical chain and substrate of the Array will be weak or impossible, a specific metal must be used to create an intermediate support layer. The metal is selected from the group comprising gold, silver, titanium or palladium due to the ability of these metals to bind with the substrate.

The specific metal, however, is chosen based on producing the strongest bond between the anchoring group, R, of the chemical chain and the intermediate support layer. In the preferred method, gold or silver is preferred for use when R is a thiol group. Titanium and other metallics are preferred when R is an amino group.

At this point, the substrate has initially been primed for the grafting of the chemical chains. As mentioned above, three methods are available as means for adding the chemical chains. In the preferred method as depicted in FIG. 9-A, a layer of resist is applied to the surface of the substrate, which includes covering the intermediate support layer, using conventional techniques. The resist may be an electron resist or photo-resist capable of being decomposed by illumination or activation by ultraviolet light, electrons, x-rays, electromagnetic fields, an acoustic source, a chemical source, a thermal source, a plasma source and an ion bombing source. The photo-resist can be a variety of nanometers thick but, in the preferred embodiment, is preferably thinner than the thickness of the SAM.

The next step involves using an illumination or activation device, including a laser operating at a wavelength that depends on the resist material. The laser is aimed at a specific location along the photo-resist covered substrate which causes decomposition of the photo-resist thus exposing the underlying pad. This process exposes at least one pad at a time which can now be used as a binding site.

In the next step, the substrate is immersed in a 10 mM solution of the specific R-T-X chain independent of the orientation to form a SAM on the exposed pad. The array is removed from solution and rinsed. The above process is repeated, i.e., using the laser to decompose the photo-resist at different points on the substrate and immersing the substrate in the different R-T-X solutions, until an Crystal Nucleating Chip is formed with a plurality of unique SAMs.

In FIG. 9-B, an alternate method for making the Crystal Nucleating Chip involves first preparing an elastomeric poly(dimethylsiloxane) (“PDMS”) stamp with a flat surface having no structural feature. If desired, the PDMS Stamp may be masked exposing specific surfaces prior to inking. However, if the substrate is masked, there is no need to mask the stamp. Further, as indicated in the steps below but for clarification, the same flat surface will be inked with an appropriate R-T-X, brought into contact, rinsed and inked again with another R-T-X, and the cycle repeated until all targeted pads are inked.

The second step involves masking the substrate using conventional lithography techniques so that at least one of the targeted pads is exposed and available for bonding with a specific chemical chain.

In the third step, the PDMS Stamp is inked with a 10 mM (milliMolar) solution of an appropriate specific chemical chain, R-T-X, brought into contact with the exposed surface on the array and removed. Both the array and the surface of the PDMS Stamp are then rinsed with copious amount of solvent to remove residual specific chemical chain, R-T-X, not adsorbed on the targeted pad area.

In the fourth step, the PDMS Stamp is horizontally oriented with the surfaces pointing downward and brought into contact with at least one exposed pad of the Array. The contact causes R, the anchoring group, to bond, either directly with the substrate or, indirectly, to the intermediate support layer, thus forming a unique self assembled monolayer on each pad. The above masking, inking and contacting process is repeated until the array is formed of highly diverse SAMS. Although this method had been modified to make the Crystal Nucleating Chip, the inking method is generally described above in the Kumar et al. article.

A third alternate method for making the array as depicted in FIG. 9C involves bonding the R-T-X linker to the pads through the anchoring functional tail group, R, and X is an amino head group. First, the amino groups are derivatized with nitroveratryloxycarbonyl (NVOC), a photo-removable protecting group.

A mask is selectively applied to the matrix of derivatized amino groups using conventional lithography techniques (FIG. 9C, STEP 3, at 4). Next, photo-deprotection of the specific targeted pad(s) is effected by illumination of the substrate through the mask, at 5. In the next step, the 1-hydroxy-benotriazole (HOBt)—activated ester of NVOC—(amino acid with different functional groups) (NVOC—amino acid with X-Obt) is allowed to react with the photodeprotected target area (pad), at 6. Next, the above steps are repeated with different target pads exposed using different masks until all pads are grafted with different kinds of amino acid in the X chain, and thus forming an Array with a plurality of chemically diverse SAMs, at 7. This method using amino groups is generally discussed in Fodor, S. P. A.; Leighton Reed, J., Pirrung, M. C,; Stryer, L.; Lu, A. T.; and Solas, D., “Light-Directed Spatially Addressable Parallel Chemical Synthesis, ”Science, 251, 767-773 (1991), which is incorporated herein by reference in its entirety.

After the array is prepared by any of the above three methods, the array is now available for immersion in the solution where crystals of the solute may form on specific SAMs.

It is to be understood that the present invention is not limited to the embodiments described above, but encompasses any and all embodiments within the scope of the following claims.

I claim:

1. A Crystal Nucleating Chip for forming crystals and determing crystal polymorphism and habit comprising:
a) a substrate;
b) a plurality of pads of approximately the same size and shape disposed on said substrate;
c) each of said pads being separated by approximately the same distance, said plurality of pads forming a pattern; and
d) a plurality of test compounds bonded to said plurality of pads, each of said pads having a different one of said test compounds bonded thereto defining a single layer self assembled monolayer on each said pad.

2. The Crystal Nucleating Chip of claim 1, further including an intermediate support layer deposited upon the substrate in order to strengthen the bonding of the plurality of test compounds to the substrate.

3. The Crystal Nucleating Chip of claim 2, wherein the intermediate support layer is a metal selected from the group consisting of gold, silver, titanium and palladium.

4. The Crystal Nucleating Chip of claim 1, wherein each said test compound has a unique chemical structure of the general formula, R-T-X, so that R and X are each separately bonded to an opposite terminal end of T, where R is an anchoring functional tail group, T is an intermediate chain group, and X is a terminal functional head group of interest.

5. The Crystal Nucleating Chip of claim 4, wherein the plurality of test compoounds are oriented substantially perpendicular to the substrate in order to maximize bonding between the substrate and the anchoring functional tail group, R.

6. The Crystal Nucleating Chip of claim 4, wherein R is an anchoring functional tail group selected from the group consisting of thiols, aminos and proteins.

7. The Crystal Nucleating Chip of claim 4, wherein T represents an intermediate chain group selected from the group consisting of CₙHₘ, CₙHₘOₚ, CₙHₘN_{q}, Si(n)Hₘ, Si(n)HₘOₚ, Si(n)HₘN_{q}, a polypeptide chain, a polynucleotide chain, SiO—SiO—SiO chain and an organometallic chain.

8. The Crystal Nucleating Chip of claim 4, wherein X represents a terminal functional head group of interest selected from the group consisting of —CH₃, —C₆H₅, —OH, —COOH, —COCH₃, —COC₂H₅, —CO(CH₃)₃, —COCH(CH₃)₂, —C(CH₃)₂OH, —CH(CH₂)₃O, —COOCH₃, —COOC₂H₅, —CH(CH₃)₂, —H, —CH₂(C₆H₅), —NH₃+, —CHCH₃OH, —SH, —CONH₂, —SCH₃, —NHC(NH₂)₂+, —C₃N₂H₄+, and —C₈NH₆.

9. A method for making a Crystal Nucleating Chip, comprising the steps of:
a) providing a substrate;
b) preparing an elastomeric poly(dimethylsiloxane) (“PDMS”) stamp with a generally flat surface;
c) masking the substrate so at least a portion of the substrate is exposed forming a pad bonded to a test compound of a unique chemical structure of a general formula, R-T-X, so that R and X are separately bonded to an opposite terminal end of T, where R is an anchoring functional tail group, T is an intermediate chain group, and X is a terminal functional head group of interest;
d) inking the elastometric PDMS Stamp with an approximately 10 mM solution of the test compound, R-T-X, in a solvent (ethanol or other solvents);
e) rinsing the surface of the PDMS stamp with copious amounts of a solvent to remove any residual specific said test compound, R-T-X;
f) grafting the test compound onto the pad by bringing the elastometric PDMS stamp and the test compound in contact with the pad; and
g) repeating the process of steps c)-f) above, until a plurality of test compounds are bonded to a plurality of pads, each of said pads having a different one of said test compounds bonded thereto defining a single layer self assembled monolayer on each said pad;
whereby the substrate forms a structure for simultaneously forming crystals and determining crystal polymorphism and habit of an inorganic or organic solute under the inducement of the plurality of test compounds, R-T-X, in a solution.

10. The method for making a Crystal Nucleating Chip of claim 9, further comprising the step of placing an intermediate support layer on said substrate.

11. The method for making a Crystal Nucleating Chip of claim 9, further comprising the step of using an intermediate support layer selected from the group consisting of gold, silver, titanium and palladium.

12. The method for making a Crystal Nucleating Chip of claim 9, further comprising the step of using an anchoring functional tail group, R, selected from the group consisting of thiols, aminos and proteins.

13. The method for making a Crystal Nucleating Chip of claim 9, further comprising the step of using an intermediate chain group, T, selected from the group consisting of CₙHₘ, CₙHₘOₚ, CₙHₘN_{q}, Si(n)Hₘ, Si(n)HₘOₚ, Si(n)HₘN_{q}, a polypeptide chain, a polynucleotide chain, SiO—SiO—SiO chain and an organometallic chain.

14. The method for making a Crystal Nucleating Chip of claim 9, further comprising the step of using a terminal functional head group of interest, X, selected from the group consisting of -CH₃, —C₆H₅, —OH, —COOH, —COCH₃, —COC₂H₅, —CO(CH₃)₃, —COCH(CH₃)₂, —C(CH₃)₂OH, —CH(CH₂)₃O, —COOCH₃, —COOC₂H₅, —CH(CH₃)₂, —H, —CH₂(C₆H₅), —NH₃+, —CHCH₃OH, —SH, —CONH₂, —SCH₃, —NHC(NH₂)₂+, —C₃N₂H₅+, and —C₈NH₆.

15. A method for using a Crystal Nucleating Chip, comprising the steps of:
a) filling a (receptacle) container with a specific solution;
b) orienting a substrate with a plurality of test compounds of a general formula R-T-X pointing substantially downward and in contact with the specific solution, where R is an anchoring functinal tail group, T is an intermediate chain group, and X is a terminal functional head group of interest;
c) removing the substrate after a specified time from the specific solution;
d) rinsing and drying the substrate;
e) inspecting the test compounds for crystallization of the specific solution using spectroscopic and non-spectroscopic techniques;
f) removing and examining at least one crystal using spectroscopic and non-spectroscopic techniques to determine crystal habit and polymorphism; and
g) using at least one crystal for further research and development and for large-scale production.

16. The method for using a Crystal Nucleating Chip of claim 15, further comprising the step of using an intermediate support layer selected from the group consisting of gold, silver, titanium and palladium.

17. The method for using a Crystal Nucleating Chip of claim 15, further comprising the step of using a plurality of test compounds with a unique chemical structure of the general formula, R-T-X, so that R and X are separately bonded to an opposite terminal end of T.

18. The method for using a Crystal Nucleating Chip of claim 15, further comprising the step of using an anchoring functional tail group, R, selected from the group consisting of thiols, aminos and proteins.

19. The method for using a Crystal Nucleating Chip of claim 15, further comprising the step of using an intermediate chain group, T, selected from the group consisting of CₙHₘ, CₙHₘOₚ, CₙHₘN_{q}, Si(n)Hₘ, Si(n)HₘOₚ, Si(n)HₘN_{q}, a polypeptide chain, a polynucleotide chain, SiO—SiO—SiO chain and an organometallic chain.

20. The method for using a Crystal Nucleating Chip of claim 15, further comprising the step of using a terminal functionalhead group of interest, X, selected from the group consisting of —CH₃, —C₆H₅, —OH, —COOH, —COCH₃, —COC₂H₅, —CO(CH₃)₃, —COCH(CH₃)₂, —C(CH₃)₂OH, —CH(CH₂)₃O, —COOCH₃, —COOC₂H₅, —CH(CH₃)₂, —H, —CH₂(C₆H₅), —NH₃+, —CHCH₃OH, —SH, —CONH₂, —SCH₃, —NHC(NH₂)₂+, —C₃N₂H₄+, and —C₈NH₆.

21. A method for making a Crystal Nucleating Chip, comprising the steps of:
a) providing a substrate;
b) depositing a photo-resist on said substrate;
c) focusing a narrow beam laser on a defined area of the photo-resist in order to decompose the photo-resist and define a pad;
d) immersing the substrate into an approximate 10 mM solution of a test compound of a general formula, R-T-X, where R is an anchoring functional tail group, T is an intermediate chain group, and X is a terminal functional head group of interest, causing the test compound to bond to the exposed pad;
e) removing and rinsing the substrate;
f) repeating steps c)to e) with an approximate 10 mM solution of a different test compound until a plurality of test compounds are bonded to a plurality of pads, each of said pads having a different one of said test compounds bonded thereto defining a single layer self assembled monolayer on each said paid;
whereby the substrate forms a structure for simultaneously forming crystals and determining crystal polymorphism and habit of an organic or inorganic solute under the inducement of the plurality of test compounds, R-T-X, in a solution.

22. The method for making a Crystal Nucleating Chip of claim further comprising the step of placing an intermediate support layer on said substrate.

23. The method for making a Crystal Nucleating Chip of claim 21, further comprising the step of using an intermediate support layer selected from the group consisting of gold, silver, titanium and palladium.

24. The method for making a Crystal Nucleating Chip of claim 21, further comprising the step of using a plurality of test compounds with a unique chemical structure of the general formula, R-T-X, so that R and X are separately bonded to an opposite terminal end of T;

25. The method for making a Crystal Nucleating Chip of claim 21, further comprising the step of using an anchoring tail group, R, selected from the group consisting of thiols, aminos and proteins.

26. The method for making a Crystal Nucleating Chip of claim 21, further comprising the step of using an intermediate chain group, T, selected from the group consisting of CₙHₘ, CₙHₘOₚ, CₙHₘN_{q}, Si(n)Hₘ, Si(n)HₘOₚ, Si(n)HₘN_{q}, a polypeptide chain, a polynucleotide chain, SiO—SiO—SiO chain and an organometallic chain.

27. The method for making a Crystal Nucleating Chip of claim 21, further comprising the step of using a terminal functional head group of interest, X, selected from the group consisting of -CH₃, —C₆H₅, —OH, —COOH, —COCH₃, —COC₂H₅, —CO(CH₃)₃, —COCH(CH₃)₂, —C(CH₃)₂OH, —CH(CH₂)₃O, —COOCH₃, —COOC₂H₅, —CH(CH₃)₂, —H, —CH₂(C₆H₅), —NH₃+, —CHCH₃OH, —SH, —CONH₂, —SCH₃, —NHC(NH₂)₂+, —C₃N₂H₄+, and —C₈NH₆.

28. A method for making a Crystal Nucleating Chip, comprising the steps of:
a) providing a substrate;
b) bonding at least one type of a NH₂-T-R linker to a pad through an anchoring functional tail group, R, to become a monolayer of NH₂-T-R-substrate;
c) derivatizing a plurality of amino groups of said monolayer of NH₂-T-R-substrate with a nitroveratryloxycarbonyl (NVOC), to form a monolayer of NVOC-NH₂-T-R-substrate;
d) applying a mask with a generally square shaped aperture on top of said monolayer of NVOC-NH₂-T-R-substrate;
e) activating (illuminating) uniformly on said mask where at least a portion of illumination passes through the generally squre shaped aperature to selectively decompose the NVOC of a portion of the monolayer of NVOC-NH₂-T-R-substrate underneath the mask and to turn said portion into said monolayer of NH₂-T-R-substrate;
f) reacting a top surface of said monolayer with a 1-hydroxylbenotriazole (HOBt)—activated ester of said NVOC—(amino acid with a different functional group, X) (NOVC—amino acid with X-Obt)to cause bonding only at a NH₂-exposed pad of the monolyaer of NH₂-T-R-substrate;
g) repeating steps c)-f) with a solution of NVOC-amino acid with X-Obt and a different X until an array of highly diverse functional groups are produced on the substrate;
whereby the array forms an analytical testing structure for simultaneously testing crystal polymorphism and habit of a solute in a solution.

29. The method of making a Crystal Nucleating Chip of claim 28, further comprising the step of using an intermediate support layer selected from the group consisting of gold, silver, titanium and palladium.

30. The method of making a Crystal Nucleating Chip of claim 28, further comprising the step of using a plurality of chemical chains with a unique chemical structure of a general formula, NH₂T-X-Obt.

31. The method of making a Crystal Nucleating Chip of claim 28, further comprising the step of selecting a functional group, X, from the group consisting of —CH₃, —C₆H₅, —OH, —COOH, —COCH₃, —COC₂H₅, —CO(CH₃)₃, —COCH(CH₃)₂, —C(CH₃)₂OH, —CH(CH₂)₃O, —COOCH₃, —COOC₂H₅, —CH(CH₃)₂, —H, —CH₂(C₆H₅), —NH₃+, —CHCH₃OH, —SH, —CONH₂, —SCH₃, —NHC(NH₂)₂+, —C₃N₂H₄+, and —C₈NH₆.

32. The method of making a Crystal Nucleating Chip of claim 28, further comprising the step of using a variety of different length (repeating) molecules, T, selected from the group consisting of CₙHₘ, CₙHₘOₚ, CₙHₘN_{q}, Si(n)Hₘ, Si(n)HₘOₚ, Si(n)HₘN_{q}, a polypeptide chain (contains C, H and N), polynucleotide chain (contains C, H, O, N and P), SiO—SiO—SiO chain and organometallic chain).

33. The method of making a Crystal Nucleating Chip of claim 28, further comprising the step of using a terminal functional molecule, X, selected from the group consisting of —CH₃, —C₆H₅, —OH, —COOH, —COCH₃, —COC₂H₅, —CO(CH₃)₃, —COCH(CH₃)₂, —C(CH₃)₂OH, —CH(CH₂)₃O, —COOCH₃, —COOC₂H₅, —CH(CH₃)₂, —H, —CH₂(C₆H₅), —NH₃+, —CHCH₃OH, —SH, —CONH₂, —SCH₃, —NHC(NH₂)₂+, —C₃N₂H₄+, and —C₈NH₆.
